(19) United States
(12) Patent Application Publication
(10) Pub. No.: US 2007/0141698 A1
(43) Pub. Date: Jun. 21, 2007

(54) Microbial protein expression system utilizing plant viral coat protein

(75) Inventors: Chung-Chi Hu, Taichung (TW); Chia-Ying Wu, Taichung (TW); Yi-Chin Lai, Taichung (TW); Hsin-Tsu Tsai, Taichung (TW); Hsin-Huang Chen, Taichung (TW)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: Bureau of Animal and Plant Health Inspection and Quarantine, Council of Agriculture, Executive Yuan

(21) Appl. No.: 11/311,197

(22) Filed: Dec. 19, 2005

(51) Int. Cl. C12P 21/06; C12N 15/00

(52) U.S. Cl. 435/320.1; 435/69.1

(57) ABSTRACT

The present invention relates to an efficient microbial protein production system. A target protein is expressed as a portion of a fusion protein with the coat protein of Cymbidium mosaic virus (CyMV) in E. coli. Accordingly, the present invention provides nucleic acid expression vectors comprising a sequence encoding a protein of interest fused to CyMV coat protein, as well as methods of utilizing such vectors to produce the protein of interest.

### BACKGROUND OF THE INVENTION

The present invention relates to the field of recombinant protein production in microbes. In particular, the present invention provides a vector encoding a target protein fused to a plant viral coat protein, and methods of utilizing the vector to express a fusion protein in bacteria.

The expression of foreign proteins in prokaryotic or eukaryotic systems is widely applied in the academic and industrial sectors. However, the efficiency for the usage of the foreign protein expression systems is heavily influenced by at least the following major characteristics of the target proteins: i) the codon usages of the particular gene; ii) the stability; iii) the toxicity; and iv) the respective purification method of the target protein.

To alleviate the above obstacles, two basic strategies have been applied to expand the usability of the foreign protein expression systems to proteins with difficulties. Firstly, bacterial strains with special features, e.g., carrying t-RNA genes for rare codons or mutations that facilitate the formation of disulfide bond formations (such as the Rosetta and Origami strains of E. coli, Novagen, EMD Biosciences, Inc., Madison, Wis., USA). Secondly, the target protein may be fused either at the N- or C-terminus with a variety of peptides or proteins, referred to as the “fusion tag,” that are designed to promote the stability and/or solubility, or to simplify the purification process. Examples of suitable commonly used fusion tags include the His tag (Crowe et al., In Methods in Molecular Biology, (Harwood, A. J., ed.), Vol. 31, pp. 371-387 (1994), Humana Press, Inc., Totawa, N.J., USA).; Sherwood, 1991), FLAG peptide (Hopp, T. P., et al., Bio/Technology 6:1204-1210 (1988)), glutathione S-transferase (GST) (Smith, D. B. et al., Gene 67:31-40 (1988)), maltose-binding protein (MBP) (Guan, C., et al., Gene, 67:21-30 (1988)), ompT/ompA (Ghrayeb, J., et al., EMBO J. 3:2437-2442 (1984)), green fluorescent protein (GFP) (Chalfie, M., et al., Science 263:802-805 (1994), avidin/streptavidin/Strep-tag (Schmidt, T. G. M. et al., Protein Eng. 6:109-122 (1993)), and NusA (Davis, G. D., et al., Biotech. Bioeng., 65:382-388 (1999).

Although the above strategies provide promising improvements, none of them alone are optimized for all target proteins. Since each protein has unique physical/chemical characteristics, it is still desirable to explore for more strategies to enhance the efficiency of the protein expression systems.

Cymbidium mosaic virus (CyMV), a member of the genus Potexvirus, is one of the most commonly seen viruses infecting orchid plants worldwide (Ajjikuttira, P. A., et al., Arch. Virol., 147:1943-54 (2002)). The coat protein of CyMV may accumulate to as high as 50 mg/ml and still maintain solubility. The CyMV virions may retain their structure and infectivity for more than 25 days at room temperature, which suggests the high stability of the CyMV coat protein.

### BRIEF SUMMARY OF THE INVENTION

The present invention relates to a fusion protein comprising a plant viral coat protein and a target protein which can be expressed with high-level in Escherichia coli.

In one aspect, the present invention relates to a nucleic acid expression vector comprising a sequence encoding a polypeptide, at least a portion of which that represents a functionally significant domain, is then fusable to a CyMV coat protein.

In another aspect, the present invention relates to a method of producing a polypeptide comprising the steps of:

(i) providing an expression vector comprising a nucleic acid sequence encoding the polypeptide, at least a portion of which that represents a functionally significant domain, is then fused to a CyMV coat protein to form a fusion protein; and

(ii) expressing the fusion protein from the vector in Escherichia coli.

In a further aspect, the present invention relates to a fusion protein comprising a polypeptide fused to a CyMV coat protein.

In yet another aspect, the present invention relates to the use of the expression vector set forth above in the production of a polypeptide in Escherichia coli.

In yet another aspect, the present invention relates to an Escherichia coli cell carrying a nucleic acid expression vector encoding a polypeptide, at least a portion of which is fused to a CyMV coat protein to form a fusion protein.

Preferably, the coat protein is the coat protein from strain CyMV-TC, that is encoded by the nucleic acid of SEQ ID NO:1 and that has the amino acid sequence of SEQ ID NO:2.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a schematic illustration of the construction of the plasmid pCy-GCP that is used to form the fusion protein. The construction of pCy-AV1c and pCy-Gemi-Rep follows the same scheme.

FIG. 2 is an image of an electorphoretic gel that shows the improvement of protein expression by fusion to the C-terminus of CyMV CP. Equal amount of proteins equivalent to 50 ml of bacterial culture were analyzed by 12.5% PAGE containing 1% SDS, followed by staining with coomassie blue. The identities of the proteins in each lane are as indicated on the top: Marker, molecular weight markers; pET21d, the vector alone without target protein, used as a negative control; pGCP 2-3-2 and pET21-AV1, non-fusion constructs of AYVV CP and AV1 genes in pET21 d, respectively; pCy-GCP and pCy-AV1c, fusion constructs of AYVV CP and AV1 genes in pCyCP-Sal, respectively. The positions of the fusion proteins, CyMV-GCP and CyMV-AV1c, are indicated on the right, with the black arrows indicating their specific positions in the image. The white arrows indicate the positions of the original, non-fusion proteins, GCP and AV1.

FIG. 3A is an image of an electorphoretic gel that shows the improvement of protein expression by fusion of various target protein portions to the C-terminus of CyMV CP. (a) Equal amount of proteins equivalent to 50 ml of bacterial culture were analyzed by 12.5% PAGE containing 1% SDS, followed by staining with coomassie blue. The identities of the proteins in each lane are as indicated on the top. The target proteins expressed in each clone are as indicated by the arrows. The white arrows indicate the positions of the original, non-fusion proteins. The black arrow indicates the position of the target protein yield from the construct pCy-Gemi-Rep. The positions of the fusion tags, CyMV CP, and glutathione, expressed from pCyCPSal or pGEX, respectively, were indicated by the “*” symbol.

FIG. 3B is a graph indicating the quantity of the target proteins that were measured by the photo-documentation system, GeneTools (Syngene Inc., Frederick, Md., USA), and displayed in a columnar chart. The Y-axis depicts the quantity of the protein in arbitrary units, and the identity of each column is shown underneath.

FIG. 4 is an image of an electorphoretic gel that shows the verification of the fusion protein by Western blot analysis with antiserum against CyMV CP; and

FIG. 5 is an image of an electorphoretic gel that shows the analysis of the solubility of the CyMV-Geminivirus CP (CyMV-GCP) fusion protein. Total proteins in the supernatant (S) and pellet (P) were analyzed by 12.5% PAGE containing 1% SDS. The identities of the proteins in each lane were as follows: Lane 1, molecular weight marker; Lane 2, supernatant proteins, grown at 37° C.; Lane 3, pellet proteins, grown at 37° C.; Lane 4, supernatant proteins in PBS containing 0.01% SDS, grown at 37° C.; Lane 5, supernatant proteins in PBS, grown at 37° C.; Lane 6, supernatant proteins, grown at 25° C.; and Lane 7, pellet proteins, grown at 25° C.

### DETAILED DESCRIPTION OF THE INVENTION

The following abbreviations are used herein:

AYVV: Ageratum yellow vein virus

CP: Coat protein

CyMV: Cymbidium mosaic virus

GCP: Geminivirus coat protein

As noted above, the present invention is directed to a protein expression system wherein a vector comprising a sequence encoding a polypeptide, at least a portion of which that represents a functionally significant domain, such as an epitope, a substrate binding site, or the activity center of enzyme, that is then fused to CyMV coat protein, is expressed in Escherichia coli. The system not only confers greater stability and solubility of the target protein, but also simplifies the purification of the target protein via an immuno-affinity technique using antibodies specific to CyMV CP.

Generally speaking, the level of recombinant protein production from a nucleic acid expression vector is influenced by a variety of factors, including but not limited to: the copy number of the vector, the strength of the promoter, the activity and localization of the recombinant protein being expressed, the host cell being used, alignment of the codon usage in the recombinant protein and host cell, and how efficiently the promoter is regulated. Not wishing to be bound by any theory, it is inferred that the unexpected compatibility of codon usage between CyMV CP and the E. coli translation system provides stable and high expression of the fusion protein.

Nucleic acid vectors useful in the present invention for cloning and expression are well known in the art, such as those described in Sambrook et al., MOLECULAR CLONING, A LABORATORY MANUAL, 2nd Ed.; Cold Spring Harbor Laboratory Press, Cold Spring Harbor, N.Y. (1989), and may include plasmids, cosmids, shuttle vectors, viral vectors, etc. Preferably, the vector used to express the fusion protein of the present invention is one conventionally used in E. coli expression systems, including but not limited to pET (Novagen, EMD Biosciences, Inc., Madison, Wis., USA), such as, for example pET21a, pET21d, pET29a, having catalog numbers 69740-3, 69743-3 69871-3, respectively; pGEX (GE Healthcare Life Science-Amersham Bioscience, Piscataway, N.J., USA), such as, for example pGEX-2T, pGEX-3X, pGEX-6P, having catalog numbers 27-4801-01, 27-4803-01, 27-4597-01, respectively; and pMAL (New England BioLabs, Inc., Ipswich, Mass., USA), such as, for example pMAL-p2G, pMAL-c2E, pMAL-c2X, having catalog numbers N8069S, N8066S, and N8076S, respectively. However, any other vector may be used for preparation of a nucleic acid expression construct as long as it is replicable and viable in E. coli.

When used herein, the term “nucleic acid” refers to any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. More particularly, the fragments are the restriction fragments generated by the digestion of appropriate restriction enzymes, such as SalI, preferably resulting in protein encoding portions or open reading frames of the nucleic acids. Nucleic acids can be either single stranded or double stranded.

Vectors useful in the expression system of the present invention comprise at least one expression control sequence (such as a promoter) and at least one cloning site (or multiple cloning sites). Such expression vectors may additionally comprise an origin of replication to ensure maintenance of the vector construct and, if desirable, to provide amplification within the host; a selectable marker permitting detectable transformation of the host cell; and a fusion tag to facilitate purification of the target protein.

The fusion protein of the invention comprises two portions: a plant viral coat protein and at least a portion of a polypeptide that represents a functionally significant domain, also known as a protein of interest (i.e., the “target” protein). The location in the fusion protein where the plant viral coat protein is joined to the target protein is referred to herein as the “fusion joint.” The fusion joint may be located at the carboxyl terminus (C-terminus) of the coat protein portion of the fusion protein (i.e., joined to the amino terminus of the target protein), or located at the amino terminus (N-terminus) of the coat protein portion of the fusion protein (i.e., joined to the carboxyl terminus of the target protein). Preferably, the fusion joint is located at the carboxyl terminus of the plant viral coat protein.

According to the present invention, the plant viral coat protein is preferably derived from CyMV, and is more preferably derived from the TC strain of CyMV, a strain of CyMV isolated from phaelanopsis orchids collected in Nan-Tou County, Taiwan, Republic of China.

To facilitate cloning, a number of amino acids may be removed from the N- or C-terminus of the CyMV coat protein by, for example, a restriction enzyme to create a cloning site for the target protein. For example, as shown in Example 1, 69 amino acids are removed from the C-terminus of CyMV coat protein to create a SalI restriction site. Knowledge of the three dimensional structure of CyMV coat protein permits persons skilled in the art, in view of the present disclosure, to determine the extent of excision and design various fusion joints without undue experimentation. Alternatively, by way of further example, one could use BstXI or ScaI, which cut at nucleotide 205 or 92, respectively, to create an appropriate fusion site.

While the polypeptide or target protein portion of the fusion protein may be derived from any of a variety of proteins, proteins for use as antigens are particularly preferred. For example, the amino acid sequence of the ageratum yellow vein virus (AYVV) CP may be used as the target protein portion of a fusion protein so as to produce a fusion protein that has antigenic properties similar to the AVYY CP. Detailed structural and functional information about many proteins are well known, and this information may be used by persons skilled in the art, in view of the present disclosure, to provide fusion proteins having the desired properties of the target protein. The target protein portion of the fusion protein may vary in size from a small number of amino acid residues, such as seventeen amino acid residues (representing the average size of the trans-membrane domain of proteins), to over several hundred amino acid residues. Preferably, the sequence of the target protein is less than 400 amino acid residues, and more preferably, the sequence of the protein of interest is less than 50 amino acid residues in length.

In one embodiment of the present invention, the fusion joint in the fusion protein is designed so as to comprise an amino acid sequence that is a substrate for a certain protease or certain poteases. By providing a fusion protein having such a fusion joint, the protein of interest can be conveniently derived from the fusion protein by using a suitable proteolytic enzyme. The proteolytic enzyme may engage the fusion protein either in vitro or in vivo.

The appropriate polypeptide-encoding nucleic acid may be inserted into the expression vector by any of a variety of well-known and routine techniques, such as, for example, those set forth in Sambrook et al., MOLECULAR CLONING, A LABORATORY MANUAL, 2nd Ed.; Cold Spring Harbor Laboratory Press, Cold Spring Harbor, N.Y. (1989).

Introduction of the expression construct into the host cell can be effected by a variety of methods with which those skilled in the art will be familiar, including but not limited to: calcium phosphate transfection, liposome-mediated transfection, transfection with naked DNA, biolistic particle-mediated transfection, DEAE-Dextran mediated transfection, or electroporation.

According to the present invention, suitable host cells are E. coli strains conventionally used in protein expression in the art. Examples include but not limited to: BL21(DE3), BL21, and Rosetta-gami (Novagen®, EMD Biosciences, Inc., Madison, Wis., USA).

Following the transformation of suitable host cells and growth of the host cells to an appropriate cell density, the expression control sequence, if it is an externally regulated promoter, is induced by appropriate means (e.g., temperature shift or chemical induction) and cells are cultured for an additional period. Cells are typically harvested by centrifugation, disrupted by physical or chemical means, and the resulting crude extract is retained for further purification. Microbial cells employed in expression of proteins can be disrupted by any convenient method, including freeze-thaw cycling, sonication, mechanical disruption, or use of cell lysing agents; and such methods are well known to those skilled in the art.

The protein expression system of the present invention provides elevated production of target proteins and thus is suitable for mass production of industrially valuable proteins, particularly proteins with antigenic properties.

### EXAMPLE 1

Virus and Bacterium Strain:

CyMV TC strain was isolated from phaelanopsis plants collected in Nan-Tou County, Taiwan, Republic of China. The complete genome of the CyMV-TC strain was sequenced using the dideoxynucleotide chain-termination method (Sanger et al., 1977, Proc. Natl. Acad. Sci. USA 74:5463-5467), and has the sequence shown in SEQ ID NO:2. Ageratum yellow vein virus (AYVV) was collected from infected Ageratum houstonianum in Ping-Dong County, Taiwan, Republic of China, and also fully sequenced.

The E. coli strain used in this and the following examples was BL21(DE3) (Novagen, EMD Biosciences, Inc., Madison, Wis., USA).

Construction of Vector Encoding the CyMV CP Fusion Protein:

The full-length CP open reading frame of CyMV CP, corresponding to nucleotides 5480 to 6151 (SEQ ID NO:1, Wu et al., unpublished), were amplified by PCR using primers CyMVCPF (5′-TACCATGGGAGAGTCC-3′, SEQ ID NO:3) and CyMVCPR (5′-TTGAGCTCTTATTCAGTAGGGGGTGC-3′, SEQ ID NO:4). The recognition sites for restriction enzymes NcoI and SacI (underlined, respectively) plus two additional nucleotides were added to the 5′ end of each primer to facilitate cloning. The PCR products were gel purified, digested with NcoI and SacI, and cloned into protein expression vector pET21 d (Novagen®, EMD Biosciences, Inc.) to give rise to pCyCP, so that full-length CyMV CP was expressed without extra amino acids from vectors. To create the unique cloning site for the insertion of target protein genes, the plasmid pCyCP was digested with SalI to remove 69 amino acids at the C-terminus (SEQ ID NO:17). At least two clones with inserts of the expected size for each construct were selected and partially sequenced by the dideoxy chain termination method (Sanger et al., 1977) to confirm the identity of the coat protein ORFs and the presence of a unique SalI site. The resulting plasmid is designated as pCyCPSal.

### EXAMPLE 2

Expression of AV1, CP, and Rep Protein of AYVV Using pCyCPSal:

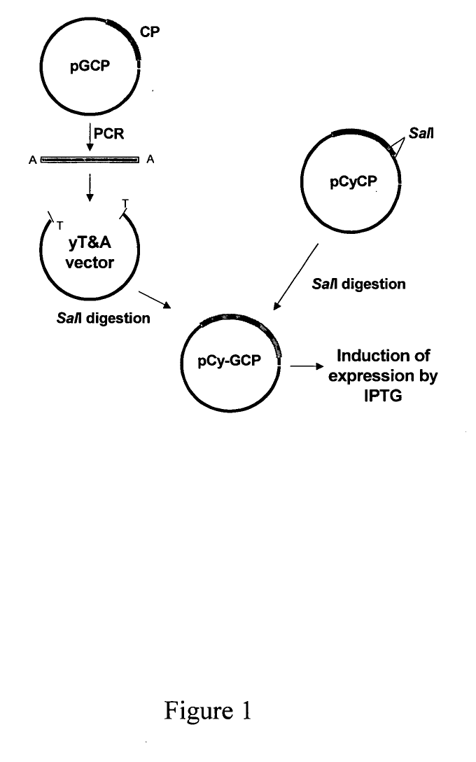

The AV1, CP, and Rep genes (SEQ ID NO:5, 7 and 9, respectively) encoding proteins (SEQ ID NO:6, 8 and 10, respectively) of AYVV were amplified by PCR using the following primer pairs, respectively: For AV1, AgAV1-SalIf: 5′-AGGTCGACTATGTGGGATCCTCTTTTGAAC-3′ (SEQ ID NO:11), AgAV1-SalIr: 5′-AAGTCGACCGGGGTTCTGTACATTCTGTAC-3′ (SEQ ID NO:12); for CP, AgCP-SalIf: 5′-AATGTCGACTATGTCGAAGCGTCCCGCAG-3′ (SEQ ID NO:13), AgCP-SalIr: 5′-AAAGTCGACCATTCTGAACAGAATCATAG-3′ (SEQ ID NO:14); and for Rep, AgRep-SalIf: 5′-TCGTCGACTATGCCTCGTTCAAG-3′ (SEQ ID NO:15), AgRep-SalIr: 5′-TCCTCGAGCGCCTGCGAACTGG-3′ (SEQ ID NO:16). The SalI site on each primer is underlined. The resulting PCR products were cloned into yT&A vetor (Yeastern Biotech, Taipei, Taiwan), digested by SalI and inserted into the unique SalI site in pCyCPSal to give rise to pCy-AV1c, pCy-GCP, and pCy-Gemi-Rep, respectively (as shown in FIG. 1).

Cultures of bacteria transformed with vector alone (pET21 d) or with CP expression plasmids were grown in LB medium (50 ml) until an OD₆₀₀ of 0.9 was reached. Expression of recombinant proteins was induced by addition of isopropyl-beta-D-thiogalactopyranoside (IPTG) to a final concentration of 1 mM. After incubation for additional 4 hrs, the cultures were placed on ice for 5 mins and the cells were collected by centrifugation at 5000 g for 5 mins at 4° C. The cells were then re-suspended in 5 ml of 10 mM Tris-HCl buffer, pH 8.0. To 7 ml of the suspension an equal volume of 2× electrophoresis loading buffer was added, and the mixture was boiled for 2 mins and analyzed by electrophoresis on 12.5% polyacryamide gels containing 1% SDS (SDS-PAGE) (Laemmli, U. K., 1970, Nature 227:680-685).

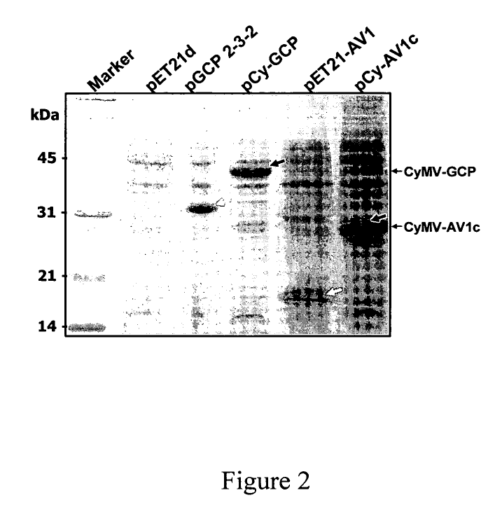

FIG. 2 is an image of an electrophoretic gel that shows the comparison of the yields of the target proteins, CP and AV1 proteins of AYVV, in the bacteria with different constructs. The same amount of total proteins extracted from bacteria harboring the following protein expression plasmids were loaded in the gel to compare the relative yields of the target proteins: pET21d, the vector alone without target protein; pGCP 2-3-2 and pET21-AV1, non-fusion constructs of AYVV CP and AV1 genes in pET21d, respectively; pCy-GCP and pCy-AV1c, fusion constructs of AYVV CP and AV1 genes in pCyCP-Sal, respectively. The target protein yields of the fusion constructs, pCy-GCP and pCy-AV1c as indicated by the black arrows, are significantly higher than the non-fusion constructs, pGCP 2-3-2 and pET21-AV1 as indicated by the white arrows, respectively. The result indicated that the yield of the target proteins, CP and AV1, can be significantly improved when fused to the C-terminus of CyMV CP.

Improvement of Protein Yield of AVYY Rep Protein:

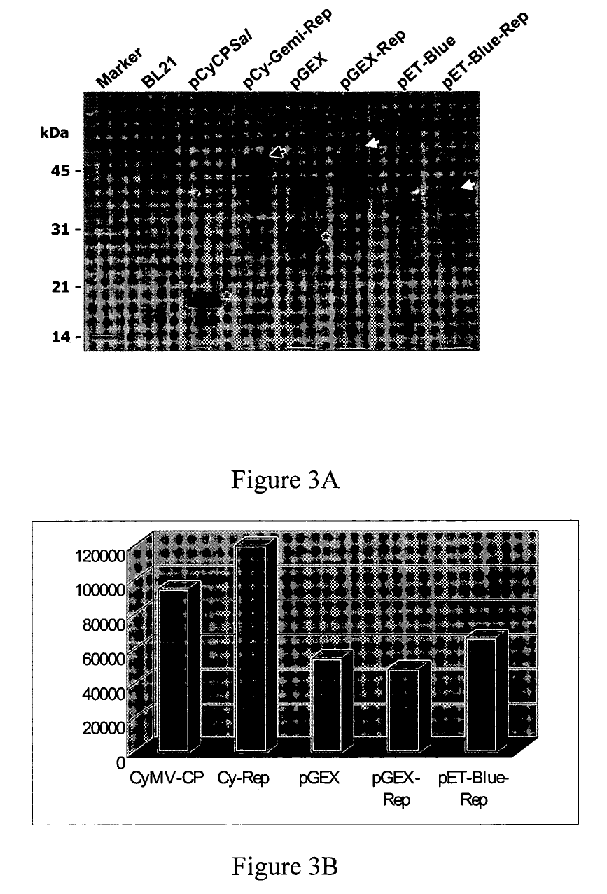

FIG. 3A is an image of an electrophoretic gel that shows another example of the improvement of protein yield of the target protein, AYVV Rep protein, when expressed from the fusion construct, pCy-Gemi-Rep. Two different expression vectors, pGEX (GE Healthcare Life Science) and pET-Blue (Novagen, EMD Biosciences, Inc., Madison, Wis., USA), were originally used to clone the Rep gene of AYVV to produce pGEX-Rep and pET-Blue-Rep, respectively. The vector pGEX and pET-Blue was chosen for the Glutathione fusion tag and the blue/white colony selection system, respectively. As shown in FIG. 3A, the target protein yield from the construct pCy-Gemi-Rep (indicated by the black arrow) is significantly higher than that for the original constructs, pGEX-Rep and pET-Blue-Rep (indicated by the white arrows). Thus, the results demonstrated the improvement of yields of various proteins by fusion to the C-terminus of CyMV CP in the vector pCyCPSal.

Western Blot Analysis:

Following SDS-PAGE (Laemmli, 1970, supra, the proteins were transferred to a PVDF membrane (Millipore Corp. Billarica, Mass., USA) with a mini trans-Blot electrophoretic transfer apparatus (Bio-Rad Laboratories, Inc., Hercules, Calif., USA) at 85 V for 1 hr. The membrane was then incubated for 1 hr at room temperature in 1×TBS (20 mM sodium Tris-HCl buffer containing 150 mM NaCl, pH 7.4) containing 0.5% non-fat milk, washed three times with 1×TBS and incubated at room temperature for 1 hr with an antiserum to target proteins diluted 1:20000 for anti-CyMV and 1:2000 for anti-Cy-GCP. After washing as above, the membrane was incubated at room temperature for 1 hr with the secondary antibody, goat anti-rabbit IgG conjugated with alkaline phosphatase (Sigma Chemical Corp., St. Louis, Mo.); diluted 1:1500 in 1×TBS containing 0.5% non-fat milk). Following washing, the target antigens were revealed by adding the buffer containing nitroblue tetrazolium (NBT) and 5-bromo-4-chloro-3-indodyl phosphate (BCIP) (Cehto et al., 1990), followed by washing with H₂O to stop the reactions and the membrane was air-dried for preservation.

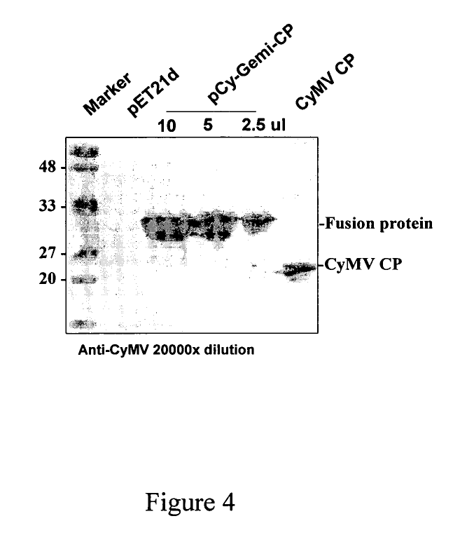

To demonstrate that the epitope structures and immunogenecity of CyMV CP are well-maintained in the fusion constructs, antiserum specific to CyMV CP was used to detect the fusion proteins by western blot analysis. FIG. 4 is an image of an electrophoretic gel that demonstrated that both the fusion protein and CyMV CP can be detected by antiserum against CyMV CP, indicating that the fusion construct did not alter or block the original epitope structures. Thus, the fusion proteins can be easily monitored, or purified by affinity columns, with a universal antiserum specific to CyMV CP. There will be no more need to produce specific antiserum to each of the target proteins.

Analysis of the Solubility of CyMV-AYVV CP Fusion Protein:

The fusion proteins were expressed at 25° C. or 37° C. in a 2 ml culture volume. After disruption of the bacterial cells, the lysates were centrifuged at 14000 rpm for 15 mins. Total proteins in the supernatant (S) and pellet (P) were analyzed on a 12.5% polyacryamide gel containing 1% SDS.

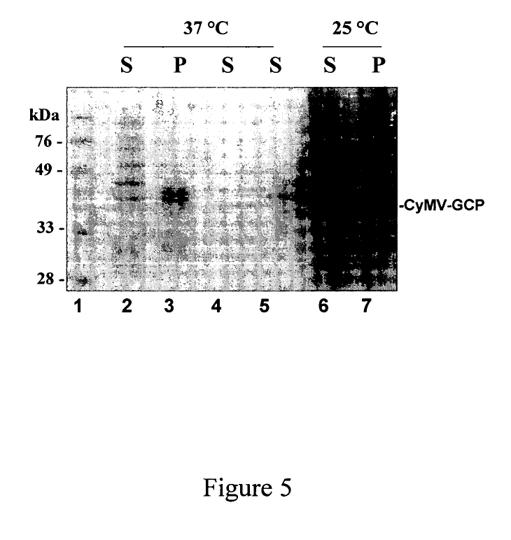

The expression conditions and solubility of the fusion protein CyMV-GCP were analyzed by SDS-PAGE (FIG. 5). After disruption of bacterial cells with ultrasonication, total bacteria lysates were centrifuged at 14000 rpm for 15 min in a bench-top centrifuge (Eppendorf 5415C). Proteins in the supernatant and pellets were analyzed by electrophoresis through a 12.5% polyacryamide gel containing 1% SDS. The proteins in the pellets were further treated with 0.01% SDS in 1× phosphate-buffered saline (1×PBS) to re-solublize the proteins. The result indicated that the bacteria can produce much more fusion proteins at 25° C. The fusion proteins expressed at 37° C. were not soluble, even after treatment of 0.01% SDS. However, the yield and solubility of the fusion proteins were greatly enhanced when expressed at 25° C. More than half of the fusion proteins are soluble. The result demonstrated the yield and solubility of the fusion protein can be significantly optimized under the right conditions.

As used herein, the article “a” or “an” means one or more than one of the grammatical object to which the article refers, unless explicitly indicated otherwise.

The disclosure of each publication referred to herein is hereby incorporated by reference herein.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A nucleic acid expression vector comprising a sequence encoding a polypeptide, at least a portion of which that represents a functionally significant domain, is then fuseable to a Cymbidium mosaic virus (CyMV) coat protein to form a fusion protein.

2. The expression vector according to claim 1, wherein the polypeptide has an N-terminus that is joined to the CyMV coat protein C-terminus.

3. The expression vector according to claim 1, wherein the coat protein is derived from CyMV-TC strain.

4. The expression vector according to claim 3, wherein the coat protein has a sequence as set forth in SEQ ID NO:2.

5. The expression vector according to claim 3, wherein the coat protein has a sequence as set forth in SEQ ID NO:17.

6. The expression vector according to claim 1, wherein the polypeptide is an antigen.

7. The expression vector according to claim 1, wherein the vector is compatible with E. coli host cells.

8. The expression vector according to claim 7, wherein the vector is pET21d.

9. A method of producing a polypeptide comprising the step of expressing a fusion protein from the expression vector of claim 1 in an E. coli host cell.

10. A method of producing a polypeptide comprising the step of expressing a fusion protein from the expression vector of claim 2 in an E. coli host cell.

11. A method of producing a polypeptide comprising the step of expressing a fusion protein from the expression vector of claim 3 in an E. coli host cell.

12. A method of producing a polypeptide comprising the step of expressing a fusion protein from the expression vector of claim 4 in an E. coli host cell.

13. A method of producing a polypeptide comprising the step of expressing a fusion protein from the expression vector of claim 5 in an E. coli host cell.

14. A fusion protein comprising a nucleic acid expression vector comprising a sequence encoding a polypeptide, at least a portion of which that represents a functionally significant domain, is then fused to a Cymbidium mosaic virus (CyMV) coat protein to form the fusion protein.

15. The fusion protein according to claim 14, wherein the polypeptide has an N-terminus that is joined to the CyMV coat protein C-terminus.

16. The fusion protein according to claim 14, wherein the coat protein is derived from CyMV-TC strain.

17. The fusion protein according to claim 16, wherein the coat protein has a sequence as set forth in SEQ ID NO:2.

18. The fusion protein according to claim 16, wherein the coat protein has a sequence as set forth in SEQ ID NO:17.

19. The fusion protein according to claim 14, wherein the polypeptide is an antigen.


SEQUENCE LISTING (17 sequences)

SEQ ID NO: 1 - DNA, 677 bp, Cymbidium mosaic virus
atgggagagtccactccgactccagctgccacctactccgctgctgaccccacttcatcacccaagctggccgacctcaccgccatcaagtactcaccggtcacctcatcaatcgccacacccgaagagatcaaggccataacccagctgtgggttaacaaccttggcctccccgctgacaccgtagtaccgcagccattgatttggctcgtgcttacgctgacgtcggggcttccaagtctgctaccctgcttggtgtcagtcccacgaagccagatgtccgccgcgccgctcttgccgcgcagatcttcgtggccaatgtcactccccgccagttttgcgcttattacggaaaagtggtgtggaatctgatgctagccaccaatgatccgcccgccaattgggccaaggctggtttccagggggatacccggtttgctgcctttgacttcttcgatgccgtcgactccaccgccgctttggaacccgctgaatggcaacgtcgccctaccgaccgtgagtgtgccgcccactcaatcgggaagtacggtgcccttgcccgacagcgcatccagagcggcaaccttatcaccaacatcgccgaggttaccaagggccatcttggctccactaacaccctctacgccctgcctggaccccctactgaataagagctc

SEQ ID NO: 2 - protein, 223 aa, Cymbidium mosaic virus
MGESTPTPAATYSAADPTSSPKLADLTAIKYSPVTSSIATPEEIKAITQLWVNNLGLPADTVVAAAIDLARAYADVGASKSATLLGVSPTKPDVRRAALAAQIFVANVTPRQFCAYYGKVVWNLMLATNDPPANWAKAGFQGDTRFAAFDFFDAVDSTAALEPAEWQRRPTDRECAAHSIGKYGALARQRIQSGNLITNIAEVTKGHLGSTNTLYALPGPPTE

SEQ ID NO: 3 - DNA, 16 bp, Artificial
  primer
taccatgggagagtcc

SEQ ID NO: 4 - DNA, 26 bp, Artificial
  primer
ttgagctcttattcagtagggggtgc

SEQ ID NO: 5 - DNA, 351 bp, Ageratum yellow vein virus
atgtgggatcctcttttgaacgagtttcctgaaactgttcacggttttaggtgtatgcttgccgtaaaatatcttcaattagttgaacagacatactctcctgatacggttggtcacggtcttatacgggatttaatttcagtaattcgtgctcgcaattatgtcgaagcgtcccgcagatattgtcatttccactcccgcctcgaaggtacgtcgccgtctgaacttcgacacccccgtgatgagccgtgctgctgcccccactgtcctcgtcaccaacagaagaaggacatggaccaacaggcccatgtaccgcaagcccagactgtacagaatgtacagaacccctga

SEQ ID NO: 6 - protein, 116 aa, Ageratum yellow vein virus
MWDPLLNEFPETVHGFRCMLAVKYLQLVEQTYSPDTVGHGLIRDLISVIRARNYVEASRRYCHFHSRLEGTSPSELRHPRDEPCCCPHCPRHQQKKDMDQQAHVPQAQTVQNVQNP

SEQ ID NO: 7 - DNA, 774 bp, Ageratum yellow vein virus
atgtcgaagcgtcccgcagatattgtcatttccactcccgcctcgaaggtacgtcgccgtctgaacttcgacacccccgtgatgagccgtgctgctgcccccactgtcctcgtcaccaacagaagaaggacatggaccaacaggcccatgtaccgcaagcccagactgtacagaatgtacagaacccctgatgtgcccaaaggttgtgaaggtccatgtaaggtacaatcgtatgaacagaggcacgacatatcccatgttggtaaggtattatgtgttagtgatgtcacccgtggtaatgggcttacacatcgtgttggtaagagattctgtgtgaagtctgtttatgtgttaggtaaaatatggatggatgaaaatattaaaactaagaaccatacgaacactgtgatgttttttcttgttcgtgacagaaggccctatggtactgctatggattttcgtcaggtgtttaacatgtatgataatgagcccagtactgctactatcaagaatgatcttcgagatcgttatcaagttttaagggaattcagttcaacagtcacagggggtcaatatgcttctaagggacaggcgtttgtggagaaatttatggagcctaacaattatgtggtttataaccatcaagaagctgctaagtatgacaatcatactgaaaatgccctgttattgtatatggcttgtactcatgccagtaatccagtgtatgctactttgaagatcagaatctatttctatgattctgttcagaattaa

SEQ ID NO: 8 - protein, 257 aa, Ageratum yellow vein virus
MSKRPADIVISTPASKVRRRLNFDTPVMSRAAAPTVLVTNRRRTWTNRPMYRKPRLYRMYRTPDVPKGCEGPCKVQSYEQRHDISHVGKVLCVSDVTRGNGLTHRVGKRFCVKSVYVLGKIWMDENIKTKNHTNTVMFFLVRDRRPYGTAMDFRQVFNMYDNEPSTATIKNDLRDRYQVLREFSSTVTGGQYASKGQAFVEKFMEPNNYVVYNHQEAAKYDNHTENALLLYMACTHASNPVYATLKIRIYFYDSVQN

SEQ ID NO: 9 - DNA, 1080 bp, Ageratum yellow vein virus
atgcctcgttcaagttcatttcaagtaaaagccgaaaatatcttcctcacttatccgaaatgcccaattcccaaagaagaagccttagagcttattaaaaatatacagtgcccttctgataaattatttatcagagttgcacaaaaaaaacactcagatgggactctgcatctccacgcgctcatacaattcaaaggtaaagcccaattcaggaatagtcgacacttcgaccttacacaccccaacacctccacacaattccatccaaacttccagggagctaagtccagttccgatgtcaagtcctacatcgagaaggacggtgattacgtcgactggggtgtatttcagatcgacggaagatctgctagaggaggtcaacagacggctaacgatgcagctgcagaagcgttaaacgcaggttcgaaagaggcagcaatggcaataattagggaaaaacttccaaaagaatatatttttcaatttcataatttaaatactaatttagataggatttttgcacctcctttagaggtatttgtttgtccttttttatcttcttcatttgatcaagttccagaagaacttgaagagtgggtttctgaaaatgtgagtggtgctgcgcggccttggagacccaaaagtattgtcatagagggagagagtcgtactggtaaaacaatgtgggccagatcactgggaccacataattatctgtgcggtcatctagatctgagtccaaaggtgtacagtaatgatgcttggtataacgtcattgatgacgtagacccccactatctaaagcactttaaagaattcatgggggcccaaagggactggcaaagcaacaccaggtacgggaaaccaattcaaattaagggtggaatcccaactatcttcctttgcaatccaggaccaacatcatcatataaagagtgcttggacgaagaaaaaaattcagcattaaaagagtgggctataaagaatgcggaattcgtcaccctcaacaacccattgtactcaggtaccaatcaaagtgcagcacaggatagcgaagaggaggccagttcgcaggcgtag

SEQ ID NO: 10 - protein, 359 aa, Ageratum yellow vein virus
MPRSSSFQVKAENIFLTYPKCPIPKEEALELIKNIQCPSDKLFIRVAQKKHSDGTLHLHALIQFKGKAQFRNSRHFDLTHPNTSTQFHPNFQGAKSSSDVKSYIEKDGDYVDWGVFQIDGRSARGGQQTANDAAAEALNAGSKEAAMAIIREKLPKEYIFQFHNLNTNLDRIFAPPLEVFVCPFLSSSFDQVPEELEEWVSENVSGAARPWRPKSIVIEGESRTGKTMWARSLGPHNYLCGHLDLSPKVYSNDAWYNVIDDVDPHYLKHFKEFMGAQRDWQSNTRYGKPIQIKGGIPTIFLCNPGPTSSYKECLDEEKNSALKEWAIKNAEFVTLNNPLYSGTNQSAAQDSEEEASSQA

SEQ ID NO: 11 - DNA, 30 bp, Artificial
  primer
aggtcgactatgtgggatcctcttttgaac

SEQ ID NO: 12 - DNA, 30 bp, Artificial
  primer
aagtcgaccggggttctgtacattctgtac

SEQ ID NO: 13 - DNA, 29 bp, Artificial
  primer
aatgtcgactatgtcgaagcgtcccgcag

SEQ ID NO: 14 - DNA, 29 bp, Artificial
  primer
aaagtcgaccattctgaacagaatcatag

SEQ ID NO: 15 - DNA, 23 bp, Artificial
  primer
tcgtcgactatgcctcgttcaag

SEQ ID NO: 16 - DNA, 22 bp, Artificial
  primer
tcctcgagcgcctgcgaactgg

SEQ ID NO: 17 - protein, 154 aa, Cymbidium mosaic virus
MGESTPTPAATYSAADPTSSPKLADLTAIKYSPVTSSIATPEEIKAITQLWVNNLGLPADTVVAAAIDLARAYADVGASKSATLLGVSPTKPDVRRAALAAQIFVANVTPRQFCAYYGKVVWNLMLATNDPPANWAKAGFQGDTRFAAFDFFDA